(19) United States
(12) Plant Patent Application Publication
(10) Pub. No.: US 2008/0313779 P1
(43) Pub. Date: Dec. 18, 2008

(54) Miniature rose plant named 'WEKmeymo'

(76) Inventor: Lawrence Meyer, Hazelwood, MO (US)

Correspondence Address: Thomas Carruth;Research Director, Weeks Wholesale Rose Grower, Inc., 4102 S. University Drive, Pomona, CA, 91768, US

(21) Appl. No.: 11/820,136

(22) Filed: Jun. 18, 2007

(51) Int. Cl. A01H 5/00

(52) U.S. Cl. PLT/122; PLT/116

(57) ABSTRACT

A new variety of miniature rose suitable for garden decoration, having single-petaled flowers of cardinal red coloration and a small white eye zone.

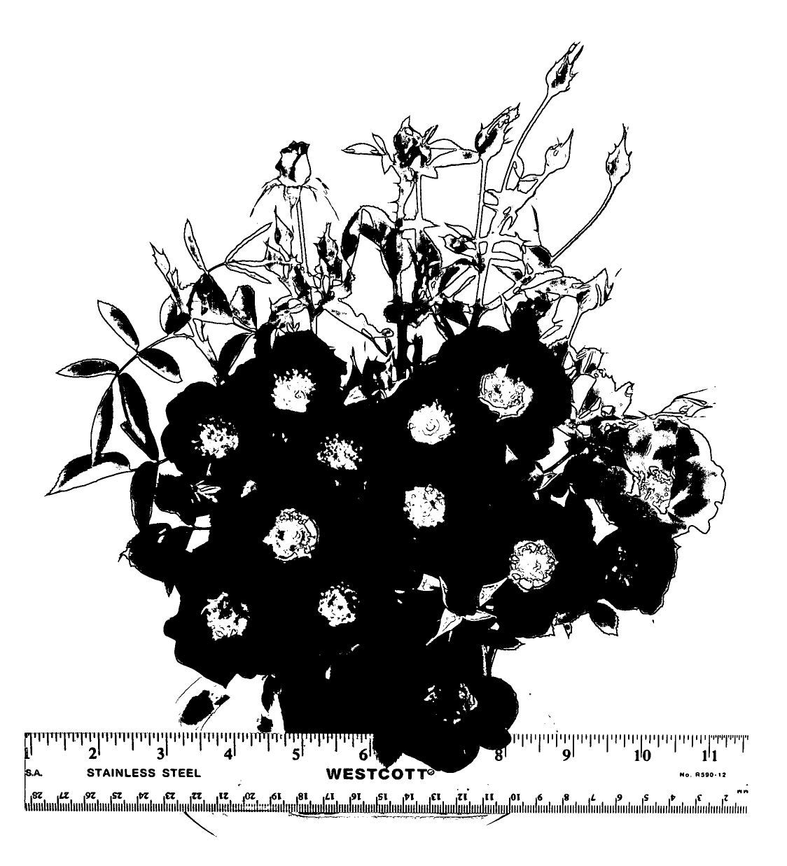

### CROSS REFERENCE

This new invention is a mutation of the miniature rose plant ‘WEKcatlart’ (United States Plant Pat. No. 12,327). This new invention is a red flowered, single petaled, miniature rose, which upon close observation can be distinguished from other red flowered, single petaled, miniature roses, as this application will set forth.

The new rose may be distinguished from ‘WEKcatlart’, which it is a mutation of, primarily by the coloring of the flowers. When grown outdoors in San Bernardino County, California, the petals of ‘WEKcatlart’ are primarily scarlet-red with a relatively large white basal area while the petals of ‘WEKmeymo’ are a darker, deep cardinal-red with noticeably less white at their base. The eye-zone marking of ‘WEKcatlart’ created by the white basal area is moderately large, covering the lower ⅓ to ½ of the petal surfaces making it very distinct, whereas the same eye zone of ‘WEKmeymo’ is smaller, not covering more than ⅕ of the basal area of the petal surfaces, and only distinguishable in the full open bloom.

### GENUS AND SPECIES

Rosa hybrida

### VARIETAL DENOMINATION

‘WEKMEYMO’

### BACKGROUND OF THE INVENTION

This invention relates to a new and distinct variety of Miniature Rose that is a mutation of ‘WEKcatlart’ (United States Plant Pat. No. 12,327). The varietal denomination of the new variety is ‘WEKmeymo’. This mutation was discovered and reproduced by Lawrence E. Meyer of Hazelwood, Mo., in October of 1999.

### SUMMARY OF THE INVENTION

Among the features which distinguish the new variety from other presently available and known commercial rose cultivars are the following combination of characteristics: its deep, non-fading cardinal-red flower color, its white eye zone noticeable on its fully open single-petaled flowers, its long lasting flowers, its bushy, fully foliated, compact plant habit that bears the flowers above the foliage, and medium-dark to dark green, semi-glossy to matte foliage. The plant has a dwarf-like, bushy, compact plant growth habit, suitable for outdoor garden decoration.

Asexual reproduction of the new variety by cuttings and by budding as performed in Hazelwood, Mo.; Kern County, California; and San Luis Obispo County, California, shows that the foregoing and other distinguishing characteristics come true to form and are established and transmitted through succeeding propagations by cuttings or budding.

### BRIEF DESCRIPTION OF ILLUSTRATION

The accompanying color photograph illustrates specimens of the new variety as grown outdoors in Upland, Calif. in the month of September and shows the flowering thereof from bud to full bloom depicted in color as nearly correct as it is possible to make in a color illustration of the character.

### DETAILED BOTANICAL DESCRIPTION

The following description is of rose plants of the new cultivar as grown outdoors in Upland Calif. The description of the actual flowers of this new cultivar is from plants grown in a greenhouse in Arroyo Grande, Calif. Phenotypic expression may vary with environmental, cultural and climatic conditions, as well as differences in conditions of light and soil. Throughout this specification, color values are based upon the Colour Chart of The Royal Horticultural Society of London, England, except where common terms of color definition are employed.

### FLOWER

The new variety mostly bears its flowers in irregular somewhat flat to slightly rounded clusters of three to four or more on strong short stems (about 6 to about 10 cm.) and also singly. Outdoors, the plant blooms very abundantly and nearly continuously during the growing season. The flowers have slight to moderate fragrance.

### BUD

The peduncle is about 2.8 to about 4 cm. in length. It is moderately rough with many stipitate glands and many very small prickles, a few reaching up to 0.24 cm. long. Peduncle color is between 144A and 146C. The side exposed to the sun is usually blushed with between 178A and 183B. The peduncle has a diameter of about 0.1 cm. to about 0.2 cm. at its widest point.

Before the calyx breaks, the bud is about 0.8 cm. to about 1.1 cm. in diameter at the widest point, about 1.7 to about 2.2 cm. in length, and pointed to ovoid in shape. The surface of the bud bears some stipitate glands, and usually with slender, entire to somewhat cut foliaceous parts extending beyond the tip of the bud about ½ or more of its length. There are 5 sepals about 1.2 cm. to about 2.6 cm. in length and about 0.4 cm. to about 0.8 cm. at the widest point. The outer surface of the sepal is a medium yellow-green, between 144A and 146C, and often blushed with between 178A and 183B, a dark color from the Greyed-Purple Group. The inner surface of the sepals is covered with fine wooly tomentum; their color is between 148B and 191B, a medium grayed-green. Sepal margins are lined with some stipitate glands and hairs.

As the petals open (after the calyx breaks), the bud is about 0.9 to about 1.2 cm. in diameter at the widest point, about 1.9 to about 2.4 cm. in length, and pointed to ovoid in form. Color when the sepals first divide is a deep cardinal red, near 53A or a mix of 53A and 53B.

### BLOOM

When fully open, the blooms range in size from about 4 to about 5 cm. in diameter and 0.4 to 0.5 cm. deep. The younger plants growing in pots under plastic usually have 5 petals and 0 to 4 petaloids. The petals are arranged regularly with 4 over-lapping (imbricated) and 1 outermost petal. Blooms from plants grown outdoors in Upland, Calif., may have 5 to 8 petals and 0 to 2 petaloids. These flowers have their 4 innermost petals imbricated and any additional petals are located outside these 4 and arranged alternatively, not overlapping.

As the blooms begin to open they are high centered with the petals loosely spiraled. Petal edges are very slightly reflexed outward and the outer margins of the petals may be somewhat sinuate. When fully open, the bloom form is more flat to slightly cupped, and the petals are somewhat flat to undulated with petal edges very slightly reflexed outward.

In September in Upland, Calif., open blooms on bushes growing outdoors generally last about five or more days. Cut roses from plants grown outdoors and kept at normal indoor living temperatures generally last about five or more days. Blooms from the younger plants grown in a greenhouse, took about 5 days to open from the time the sepals first separated, and an additional 4 to 5 days until discoloration began. On the spent bloom, the petals usually drop off cleanly.

### PETALS

The substance of the petals is very heavy and very thick with upper surfaces moderately velvety to satiny and under surfaces more shiny to satiny. The petals are nearly round to broadly obovate in shape with cuspidate apices. The petal margins are usually entire or may be somewhat crenate. The basal area comes about ⅕ of the way up the petal from the base.

### NEWLY OPENED FLOWER

The color of the under surfaces of the newly opened petals is a deep cardinal red, near 53A or a mix of near 53A and near 53B. The basal area is white, near 155B and becoming a very light Pea Green, near 149D, approaching and at the point of attachment. There are often streaks of French Rose, near 49D, from the basal area to the apex of the petal. These streaks do not show through to the upper surface. The color of the upper surfaces of the newly opened petals is near 53A, suffused with varying degrees of near 187A from the Greyed-Purple Group. There may be areas of varying sizes or occasional stripes of red coloring similar to the color of ‘WEKcatlart’ from which it sported. The basal area is white, nearest 155A, and the point of attachment is near 150D, Sap Green.

### THREE DAY OLD FLOWER

Coloring changes very little until the fourth or fifth day after the flower begins to open. At that point, the under surface becomes darker with a blend of near 187C and 187D. Any streaks on the under surfaces going from the basal area to the apex are still near 49D. The basal area whitens to near 155D. The color of upper surface deepens to near 187B with near 187A along the outer margins and graduating to near 59C, from the Red-Purple Group, right above the basal area. The basal area becomes whiter, near 155D.

### PETALOIDS

Petaloids, when present, are as the size and shape of a petal cut in half vertically. The coloring is similar to that of the petals but with a strip of white, near 155A, up along one side. The basal area and point of attachment are near 153C, a medium yellow green. The base of petaloids is often linear, very narrow, down to the point of attachment.

### MALE REPRODUCTIVE ORGANS

Stamens are average to many in number (varying from 50 to 80) and are arranged regularly about the pistils. The filaments are irregular in length, most with anthers. Filaments are a medium yellow, between 10A and 14C in color. The anthers are moderately large for the class and all open approximately at the same time. Anther color is a darker yellow, near 14B, when immature and near 164A, from the Greyed-Orange Group, at maturity. Pollen is very abundant and light yellow, near 13D, in color.

### FEMALE REPRODUCTIVE ORGANS

Pistils vary in number, usually between 30 to 45. The styles are somewhat uneven and loosely bunched. Stigma and style color is near between 1C and 4C, a medium to light greenish yellow. Ovaries are usually all enclosed in the calyx.

Hips on this variety when grown in Upland, Calif. are moderately globular in form, and near 28A, Persimmon Orange, in color when ripe. The hip surface is very smooth with somewhat thick fleshy walls. The sepals are moderately fugacious.

### FOLIAGE

The compound leaves are usually comprised of three to five or more leaflets and are borne very abundantly. The five leaflet leaves are about 4.8 to about 6.5 cm. in length and about 3.5 to about 4.5 cm. in width at the widest point, and moderately semi-glossy to matte in finish. Leaflets are moderately heavy in texture. The venation pattern is pinnate . The color of the veins is a very dark green, between 147B and 139B. The terminal leaflets are about 2.1 to about 2.6 cm. in length and about 1.4 to about 1.7 cm. in width at the widest point, oval to somewhat ovate in shape with acute apices and somewhat round to moderately acute bases. Their margins are usually simply serrate.

The petiole is about 0.8 to 1.1 cm. in length and less than about 0.1 cm. in diameter at the widest point. The petiolule is about 0.1 to about 0.2 cm. in length and less than about 0.1 cm. in diameter at the widest point. Their color is a medium yellow-green, between 144A and 144C, and sometimes lightly suffused with a dark, grayed red-purple, between 178A and 183B.

The upper surface of the mature leaf is a very dark green, between 137A and 139A. The under surface of the mature leaf is lighter, between 147B and 191A. The upper surface of the young leaf is a Lettuce Green, near 144A, sometimes lightly suffused with between 178A and 183B. The under surface of the young leaf is a medium yellow-green, between 149C and 138B, often lightly suffused with between 178A and 183B.

The rachis is moderately smooth. The upper side is shallowly grooved with some stipitate glands on the edges of the grooves. The under side of the rachis is very smooth with few stipitate glands and very few small prickles.

The stipules are about 0.4 to about 0.6 cm. in length and of medium width with straight points that usually turn out at an angle of less than 45 degrees. The color is between 144A and 144C, sometimes lightly suffused with between 178A and 183B.

The plant displays an above average degree of resistance to powdery mildew and rust as compared to other commercial varieties grown under comparable conditions in Upland, Calif.

### GROWTH

The plant has a dwarf-like, bushy, compact, low to medium height growth habit (about 28 to about 42 cm. in height and spread at the widest point), with very full branching. It displays very vigorous growth. Cuttings usually take 4 to 10 days to initiate root development under controlled greenhouse conditions.

The following colors and measurements were taken from mature plants grown outside in Upland, Calif:

The color of the major stems is a medium yellow-green, between 147C and 138B. They bear many large prickles that are about 0.6 to about 0.8 cm. in length. The large prickles are very straight and angled slightly downward with a moderately long narrow base; prickle color is a medium brown, between 165B and 166C, from the Greyed-Orange Group. The major stem bears several small prickles of varying sizes but of similar shape and coloration.

The color of the branches is a little darker than that of the major stems, between 147B and 138A. They bear many large prickles that are about 0.6 to about 0.8 cm. in length. The large prickles are very straight and angled slightly downward with a moderately long narrow base; prickle color is between 180A and 178C, a medium color from the Greyed-Red Group. The major stem bears several small prickles of varying sizes but of similar shape and coloration.

The color of the new shoots is near 144A and often suffused with between 178A and 183B. They bear many large prickles that are about 0.6 to about 0.8 cm. in length. The large prickles are very straight and angled slightly downward with a moderately long narrow base; prickle color is between 178A and 183A. The major stem bears several small prickles of varying sizes but of similar shape and coloration.

Winter hardiness and heat tolerance are still being determined.

1. A new and distinct variety of hardy, miniature rose plant is claimed, substantially as described and illustrated herein.
